(19) United States
(12) Patent Application Publication
(10) Pub. No.: US 2022/0379127 A1
(43) Pub. Date: Dec. 1, 2022

(54) Forecasting Stimulation Adjustments in a Stimulator System Using Time Series Analysis

(71) Applicant: Boston Scientific Neuromodulation Corporation, Valencia, CA (US)

(72) Inventors: Ismael Huertas Fernandez, Sevilla (ES); Que T. Doan, West Hills, CA (US)

(21) Appl. No.: 17/663,491

(22) Filed: May 16, 2022

Related U.S. Application Data
(60) Provisional application No. 63/193,164, filed on May 26, 2021.

(51) Int. Cl. A61N 1/372; A61N 1/36; A61N 1/378

(52) U.S. Cl. CPC A61N 1/37252; A61N 1/36062; A61N 1/36071; A61N 1/378

(57) ABSTRACT

Systems and methods are disclosed in which a time series analysis algorithm is used to analyze inputs such as adjustments a patient has made to the amplitude of stimulation in an implantable stimulator system. The algorithm uses these inputs to predict how the patient would likely adjust the amplitude in the future, i.e. to predict future amplitudes for the patient as a function of time. Preferably, the algorithm determines one or more of an amplitude level, at least one seasonal variation, or at least one trend when predicting the amplitude. This predicted amplitude can then be used to automatically adjust the amplitude of the stimulation provided by the patient's stimulator. The algorithm may only use previous amplitude adjustments to predict the amplitude, other time-varying inputs, or combinations of both.

### CROSS REFERENCE TO RELATED APPLICATIONS

This is a non-provisional application of U.S. Provisional Patent Application Ser. No. 63/193,164, filed May 26, 2021, which is incorporated herein by reference, and to which priority is claimed.

### FIELD OF THE INVENTION

This application relates to Implantable Medical Devices (IMDs) generally, Spinal Cord Stimulators more specifically, and to methods of control of such devices.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability with any implantable neurostimulator device system.

An SCS system typically includes an Implantable Pulse Generator (IPG) 10 shown in FIG. 1. The IPG 10 includes a biocompatible device case 12 that holds the circuitry and battery 14 necessary for the IPG to function. The IPG 10 is coupled to electrodes 16 via one or more electrode leads 15 that form an electrode array 17. The electrodes 16 are configured to contact a patient's tissue and are carried on a flexible body 18, which also houses the individual lead wires 20 coupled to each electrode 16. The lead wires 20 are also coupled to proximal contacts 22, which are insertable into lead connectors 24 fixed in a header 23 on the IPG 10, which header can comprise an epoxy for example. Once inserted, the proximal contacts 22 connect to header contacts within the lead connectors 24, which are in turn coupled by feedthrough pins through a case feedthrough to circuitry within the case 12, although these details aren't shown.

In the illustrated IPG 10, there are sixteen lead electrodes (E1-E16) split between two leads 15, with the header 23 containing a 2×1 array of lead connectors 24. However, the number of leads and electrodes in an IPG is application specific and therefore can vary. The conductive case 12 can also comprise an electrode (Ec). In a SCS application, the electrode leads 15 are typically implanted proximate to the dura in a patient's spinal column on the right and left sides of the spinal cord midline. The proximal electrodes 22 are tunneled through the patient's tissue to a distant location such as the buttocks where the IPG case 12 is implanted, at which point they are coupled to the lead connectors 24. In other IPG examples designed for implantation directly at a site requiring stimulation, the IPG can be lead-less, having electrodes 16 instead appearing on the body of the IPG for contacting the patient's tissue. The IPG leads 15 can be integrated with and permanently connected the case 12 in other IPG solutions. The goal of SCS therapy is to provide electrical stimulation from the electrodes 16 to alleviate a patient's symptoms, most notably chronic back pain.

IPG 10 can include an antenna 26a allowing it to communicate bi-directionally with a number of external devices, as shown in FIG. 4. The antenna 26a as depicted in FIG. 1 is shown as a conductive coil within the case 12, although the coil antenna 26a can also appear in the header 23. When antenna 26a is configured as a coil, communication with external devices preferably occurs using near-field magnetic induction. IPG may also include a Radio-Frequency (RF) antenna 26b. In FIG. 1, RF antenna 26b is shown within the header 23, but it may also be within the case 12. RF antenna 26b may comprise a patch, slot, or wire, and may operate as a monopole or dipole. RF antenna 26b preferably communicates using far-field electromagnetic waves. RF antenna 26b may operate in accordance with any number of known RF communication standards, such as Bluetooth, Zigbee, WiFi, MICS, and the like.

Stimulation in IPG 10 is typically provided by pulses, as shown in FIG. 2. Stimulation parameters typically include the amplitude of the pulses (I; whether current or voltage); the frequency (F) and pulse width (PW) of the pulses; the electrodes 16 (E) activated to provide such stimulation; and the polarity (P) of such active electrodes, i.e., whether active electrodes are to act as anodes (that source current to the tissue) or cathodes (that sink current from the tissue). These stimulation parameters taken together comprise a stimulation program that the IPG 10 can execute to provide therapeutic stimulation to a patient.

In the example of FIG. 2, electrode E5 has been selected as an anode, and thus provides pulses which source a positive current of amplitude +I to the tissue. Electrode E4 has been selected as a cathode, and thus provides pulses which sink a corresponding negative current of amplitude −I from the tissue. This is an example of bipolar stimulation, in which only two lead-based electrodes are used to provide stimulation to the tissue (one anode, one cathode). However, more than one electrode may act as an anode at a given time, and more than one electrode may act as a cathode at a given time (e.g., tripole stimulation, quadripole stimulation, etc.).

The pulses as shown in FIG. 2 are biphasic, comprising a first phase 30a, followed quickly thereafter by a second phase 30b of opposite polarity. As is known, use of a biphasic pulse is useful in active charge recovery. For example, each electrodes' current path to the tissue may include a serially-connected DC-blocking capacitor, see, e.g., U.S. Patent Application Publication 2016/0144183, which will charge during the first phase 30a and discharged (be recovered) during the second phase 30b. In the example shown, the first and second phases 30a and 30b have the same duration and amplitude (although opposite polarities), which ensures the same amount of charge during both phases. However, the second phase 30b may also be charged balance with the first phase 30a if the integral of the amplitude and durations of the two phases are equal in magnitude, as is well known. The width of each pulse, PW, is defined here as the duration of first pulse phase 30a, although pulse width could also refer to the total duration of the first and second pulse phases 30a and 30b as well. Note that an interphase period (IP) during which no stimulation is provided may be provided between the two phases 30a and 30b.

IPG 10 includes stimulation circuitry 28 that can be programmed to produce the stimulation pulses at the electrodes as defined by the stimulation program. Stimulation circuitry 28 can for example comprise the circuitry described in U.S. Patent Application Publications 2018/0071513 and 2018/0071520, or described in U.S. Pat. Nos. 8,606,362 and 8,620,436. These references are incorporated herein by reference.

FIG. 3 shows an external trial stimulation environment that may precede implantation of an IPG 10 in a patient. During external trial stimulation, stimulation can be tried on a prospective implant patient without going so far as to implant the IPG 10. Instead, one or more trial leads 15′ are implanted as an electrode array 17 in the patient's tissue 32 at a target location, such as within the spinal column as explained earlier. The proximal ends of the trial lead(s) 15′ exit an incision 36 and are connected to an External Trial Stimulator (ETS) 40. The ETS 40 generally mimics operation of the IPG 10, and thus can provide stimulation pulses to the patient's tissue as explained above. See, e.g., U.S. Pat. No. 9,259,574, disclosing a design for an ETS. The ETS 40 is generally worn externally by the patient for a short while (e.g., two weeks), which allows the patient and his clinician to experiment with different stimulation parameters to try and find a stimulation program that alleviates the patient's symptoms (e.g., pain). If external trial stimulation proves successful, trial lead(s) 15′ are explanted, and a full IPG 10 and lead(s) 15 are implanted as described above; if unsuccessful, the trial lead(s) 15′ are simply explanted.

Like the IPG 10, the ETS 40 can include one or more antennas to enable bi-directional communications with external devices, explained further with respect to FIG. 4. Such antennas can include a near-field magnetic-induction coil antenna 42a, and/or a far-field RF antenna 42b, as described earlier. ETS 40 may also include stimulation circuitry 44 able to form the stimulation pulses in accordance with a stimulation program, which circuitry may be similar to or comprise the same stimulation circuitry 28 present in the IPG 10. ETS 40 may also include a battery (not shown) for operational power.

FIG. 4 shows various external devices that can wirelessly communicate data with the IPG 10 and the ETS 40, including a patient, hand-held external controller 45, and a clinician programmer 50. Both of devices 45 and 50 can be used to send a stimulation program to the IPG 10 or ETS 40—that is, to program their stimulation circuitries 28 and 44 to produce pulses with a desired shape and timing described earlier. Both devices 45 and 50 may also be used to adjust one or more stimulation parameters of a stimulation program that the IPG 10 or ETS 40 is currently executing. Devices 45 and 50 may also receive information from the IPG 10 or ETS 40, such as various status information, etc.

External controller 45 can be as described in U.S. Patent Application Publication 2015/0080982 for example, and may comprise either a dedicated controller configured to work with the IPG 10. External controller 45 may also comprise a general purpose mobile electronics device such as a mobile phone which has been programmed with a Medical Device Application (MDA) allowing it to work as a wireless controller for the IPG 10 or ETS 40, as described in U.S. Patent Application Publication 2015/0231402. External controller 45 includes a user interface, including means for entering commands (e.g., buttons or icons) and a display 46. The external controller 45's user interface enables a patient to adjust stimulation parameters, although it may have limited functionality when compared to the more-powerful clinician programmer 50, described shortly.

The external controller 45 can have one or more antennas capable of communicating with the IPG 10 and ETS 40. For example, the external controller 45 can have a near-field magnetic-induction coil antenna 47a capable of wirelessly communicating with the coil antenna 26a or 42a in the IPG 10 or ETS 40. The external controller 45 can also have a far-field RF antenna 47b capable of wirelessly communicating with the RF antenna 26b or 42b in the IPG 10 or ETS 40.

The external controller 45 can also have control circuitry 48 such as a microprocessor, microcomputer, an FPGA, other digital logic structures, etc., which is capable of executing instructions an electronic device. Control circuitry 48 can for example receive patient adjustments to stimulation parameters, and create a stimulation program to be wirelessly transmitted to the IPG 10 or ETS 40.

Clinician programmer 50 is described further in U.S. Patent Application Publication 2015/0360038, and is only briefly explained here. The clinician programmer 50 can comprise a computing device 51, such as a desktop, laptop, or notebook computer, a tablet, a mobile smart phone, a Personal Data Assistant (PDA)-type mobile computing device, etc. In FIG. 4, computing device 51 is shown as a laptop computer that includes typical computer user interface means such as a screen 52, a mouse, a keyboard, speakers, a stylus, a printer, etc., not all of which are shown for convenience. Also shown in FIG. 4 are accessory devices for the clinician programmer 50 that are usually specific to its operation as a stimulation controller, such as a communication “wand” 54, and a joystick 58, which are coupleable to suitable ports on the computing device 51, such as USB ports 59 for example.

The antenna used in the clinician programmer 50 to communicate with the IPG 10 or ETS 40 can depend on the type of antennas included in those devices. If the patient's IPG 10 or ETS 40 includes a coil antenna 26a or 42a, wand 54 can likewise include a coil antenna 56a to establish near-filed magnetic-induction communications at small distances. In this instance, the wand 54 may be affixed in close proximity to the patient, such as by placing the wand 54 in a belt or holster wearable by the patient and proximate to the patient's IPG 10 or ETS 40.

If the IPG 10 or ETS 40 includes an RF antenna 26b or 42b, the wand 54, the computing device 51, or both, can likewise include an RF antenna 56b to establish communication with the IPG 10 or ETS 40 at larger distances. (Wand 54 may not be necessary in this circumstance). The clinician programmer 50 can also establish communication with other devices and networks, such as the Internet, either wirelessly or via a wired link provided at an Ethernet or network port.

To program stimulation programs or parameters for the IPG 10 or ETS 40, the clinician interfaces with a clinician programmer graphical user interface (GUI) 64 provided on the display 52 of the computing device 51. As one skilled in the art understands, the GUI 64 can be rendered by execution of clinician programmer software 66 on the computing device 51, which software may be stored in the device's non-volatile memory 68. One skilled in the art will additionally recognize that execution of the clinician programmer software 66 in the computing device 51 can be facilitated by control circuitry 70 such as a microprocessor, microcomputer, an FPGA, other digital logic structures, etc., which is capable of executing programs in a computing device. Such control circuitry 70, in addition to executing the clinician programmer software 66 and rendering the GUI 64, can also enable communications via antennas 56a or 56b to communicate stimulation parameters chosen through the GUI 64 to the patient's IPG 10.

A portion of the GUI 64 is shown in one example in FIG. 5. One skilled in the art will understand that the particulars of the GUI 64 will depend on where clinician programmer software 66 is in its execution, which will depend on the GUI selections the clinician has made. FIG. 5 shows the GUI 64 at a point allowing for the setting of stimulation parameters for the patient and for their storage as a stimulation program. To the left a program interface 72 is shown, which as explained further in the '038 Publication allows for naming, loading and saving of stimulation programs for the patient. Shown to the right is a stimulation parameters interface 82, in which specific stimulation parameters (I, D, F, E, P, X) can be defined for a stimulation program. Values for stimulation parameters relating to the shape of the waveform (I; in this example, current), pulse width (PW), and frequency (F) are shown in a waveform parameter interface 84, including buttons the clinician can use to increase or decrease these values.

Stimulation parameters relating to the electrodes 16 (the electrodes E activated and their polarities P), are made adjustable in an electrode parameter interface 86. Electrode stimulation parameters are also visible and can be manipulated in a leads interface 92 that displays the leads 15 (or 15′) in generally their proper position with respect to each other, for example, on the left and right sides of the spinal column. A cursor 94 (or other selection means such as a mouse pointer) can be used to select a particular electrode in the leads interface 92. Buttons in the electrode parameter interface 86 allow the selected electrode (including the case electrode, Ec) to be designated as an anode, a cathode, or off. The electrode parameter interface 86 further allows the relative strength of anodic or cathodic current of the selected electrode to be specified in terms of a percentage, X. This is particularly useful if more than one electrode is to act as an anode or cathode at a given time, as explained in the '038 Publication. In accordance with the example waveforms shown in FIG. 2, as shown in the leads interface 92, electrode E5 has been selected as the only anode to source current, and this electrode receives X=100% of the specified anodic current, +A. Likewise, electrode E4 has been selected as the only cathode to sink current, and this electrode receives X=100% of that cathodic current, −A. As such, stimulation is this example is provided as a bipole, with one anode pole (+) and one cathode pole (−) being formed in the electrode array 17. Other pole configurations (e.g., tripoles) can be formed as well, and the locations of the pole may appear at random locations relative to the physical positions of the electrodes, as is known. Further, through adjustment of percentage X, poles may be configured that are virtual, and that have positions that do not necessarily correspond to the physical positions of the electrodes, as explained further in U.S. Pat. No. 10,881,859.

The GUI 64 as shown specifies only a pulse width PW of the first pulse phase 30a. The clinician programmer software 66 that runs and receives input from the GUI 64 will nonetheless ensure that the IPG 10 and ETS 40 are programmed to render the stimulation program as biphasic pulses if biphasic pulses are to be used. For example, the clinician programming software 66 can automatically determine durations and amplitudes for both of the pulse phases 30a and 30b (e.g., each having a duration of PW, and with opposite polarities +I and −I). An advanced menu 88 can also be used (among other things) to define the relative durations and amplitudes of the pulse phases 30a and 30b, and to allow for other more advance modifications, such as setting of a duty cycle (on/off time) for the stimulation pulses, and a ramp-up time over which stimulation reaches its programmed amplitude (I), etc. A mode menu 90 allows the clinician to choose different modes for determining stimulation parameters. For example, as described in the '038 Publication, mode menu 90 can be used to enable electronic trolling, which comprises an automated programming mode that performs current steering along the electrode array by moving the cathode in a bipolar fashion.

While GUI 64 is shown as operating in the clinician programmer 50, the user interface of the external controller 45 may provide similar functionality.

### SUMMARY

A system is disclosed, which may comprise: an external device for controlling a stimulator device that provides stimulation to a patient, the external device comprising: a user interface configured to receive adjustments to a stimulation parameter of the stimulation provided by the stimulator device; and controller circuitry programmed with an algorithm, wherein the algorithm is configured to: analyze the adjustments to the stimulation parameter to determine a forecasted stimulation parameter, wherein the forecasted stimulation parameter comprises a periodic variation as a function of time or a non-periodic trend as a function of time; and automatically control the stimulator device over time to provide the stimulation in accordance with the forecasted stimulation parameter.

In one example, the algorithm comprises a time series analysis algorithm. In one example, the time series analysis algorithm comprises a seasonal autoregressive integrated moving average algorithm. In one example, the adjusted stimulation parameter and the forecasted stimulation parameter comprise a stimulation amplitude. In one example, the forecasted stimulation parameter comprises a sum of the periodic variation and the non-periodic trend. In one example, the forecasted stimulation parameter comprises a product of the periodic variation and the non-periodic trend. In one example, the forecasted stimulation parameter further comprises a time-invariant level. In one example, the forecasted stimulation parameter comprises a function of the periodic variation, the non-periodic trend, and the time-invariant level. In one example, the external device comprises a portable patient external controller. In one example, the forecasted stimulation parameter comprises the periodic variation as a function of time and the non-periodic trend as a function of time. In one example, the external device comprises telemetry circuitry configured to transmit the forecasted stimulation parameter to the stimulator device. In one example, the stimulator device is automatically controlled in accordance with the forecasted stimulation parameter by adjusting that stimulation parameter at the stimulator device as a function of time. In one example, the user interface comprises a selectable option to cause the external device to automatically control the stimulator device to provide the stimulation in accordance with the forecasted stimulation parameter. In one example, the user interface comprises at least one option to permit the patient to override automatic control of the stimulator device by manually controlling the stimulation parameter. In one example, the external device is configured to revert to automatic control of the stimulator device a time period after manual control of the stimulation parameter. In one example, the system of claim 1, further comprises the stimulator device. In one example, the stimulator device is configured to provide the stimulation as electrical stimulation at one or more electrodes of the stimulator device. In one example, the stimulator device is implantable in the patient.

A method is disclosed using an external device for controlling a stimulator device that provides stimulation to a patient. The method may comprise: receiving at the external device adjustments to a stimulation parameter of the stimulation provided by the stimulator device; analyzing at the external device the adjustments to the stimulation parameter to determine a forecasted stimulation parameter, wherein the forecasted stimulation parameter comprises a periodic variation as a function of time or a non-periodic trend as a function of time; and automatically controlling the stimulator device over time to provide the stimulation in accordance with the forecasted stimulation parameter.

In one example, analyzing the adjustment to the stimulation parameter comprises use of a time series analysis algorithm. In one example, the time series analysis algorithm comprises a seasonal autoregressive integrated moving average algorithm. In one example, the adjusted stimulation parameter and the forecasted stimulation parameter comprise a stimulation amplitude. In one example, the forecasted stimulation parameter comprises a sum of the periodic variation and the non-periodic trend. In one example, the forecasted stimulation parameter comprises a product of the periodic variation and the non-periodic trend. In one example, the forecasted stimulation parameter further comprises a time-invariant level. In one example, the forecasted stimulation parameter comprises a function of the periodic variation, the non-periodic trend, and the time-invariant level. In one example, the external device comprises a portable patient external controller. In one example, the forecasted stimulation parameter comprises the periodic variation as a function of time and the non-periodic trend as a function of time. In one example, the external device transmits the forecasted stimulation parameter to the stimulator device to automatically control the stimulator device in accordance with the forecasted stimulation parameter. In one example, the stimulator device is automatically controlled in accordance with the forecasted stimulation parameter by adjusting that stimulation parameter at the stimulator device as a function of time. In one example, the adjustments to the stimulation parameter are received at a user interface of the external device. In one example, the user interface comprises a selectable option to cause the external device to automatically control the stimulator device to provide the stimulation in accordance with the forecasted stimulation parameter. In one example, the method further comprises overriding automatic control of the stimulator device by receiving at least one input at the user interface to manually control the stimulation parameter. In one example, the method further comprises reverting to automatic control of the stimulator device a time period after manual control of the stimulation parameter. In one example, the stimulator device provides the stimulation as electrical stimulation at one or more electrodes of the stimulator device. In one example, the stimulator device is implantable in the patient.

A non-transitory computer readable medium is disclosed which may comprise instructions executable on an external device for controlling a stimulator device that provides stimulation to a patient, wherein the instructions when executed enable the external device to: receive adjustments to a stimulation parameter of the stimulation provided by the stimulator device; analyze the adjustments to the stimulation parameter to determine a forecasted stimulation parameter as a function of time, wherein the forecasted stimulation parameter comprises a periodic variation as a function of time or a non-periodic trend as a function of time; and automatically control the stimulator device over time to provide the stimulation in accordance with the forecasted stimulation parameter.

### BRIEF DESCRIPTION OF THE DRAWINGS

FIG. 1 shows an Implantable Pulse Generator (IPG) useable for Spinal Cord Stimulation (SCS), in accordance with the prior art.

FIG. 2 shows an example of stimulation pulses producible by the IPG, in accordance with the prior art.

FIG. 3 shows use of an External Trial Stimulator (ETS) useable to provide stimulation before implantation of an IPG, in accordance with the prior art.

FIG. 4 shows various external devices capable of communicating with and programming stimulation in an IPG and ETS, in accordance with the prior art.

FIG. 5 shows a Graphical User Interface (GUI) of a clinician programmer external device for setting or adjusting stimulation parameters, in accordance with the prior art.

FIG. 6 shows a patient's adjustment to stimulation amplitude, and shows a general amplitude level and seasonal (periodic) variations over the course of a day.

FIG. 7 shows how a patient's adjustment to stimulation can follow a non-periodic trend as a function of time.

FIGS. 8A-8B show use of a time series analysis algorithm to forecast an amplitude for the patient that varies as a function of time using previous patient adjustments to stimulation amplitude.

FIG. 9 shows an implementation of the algorithm within an external device, and use of the forecasted amplitude to automatically adjust stimulation amplitude in the patient's IPG or ETS.

FIG. 10 shows a graphical user interface of the external device that provides information about operation of the algorithm, and which allows the patient to manually adjust amplitude to override automatic control of amplitude.

FIG. 11 shows modification to the time series analysis algorithm, and in particular shows additional inputs to the algorithm that may be considered.

FIG. 12 shows an example in which the input of perception threshold (pth) is used to modify operation of the algorithm.

FIG. 13 shows modification to the time series analysis algorithm, and in particular shows the use of only other inputs to the algorithm, but not previous patient adjustments to stimulation amplitude.

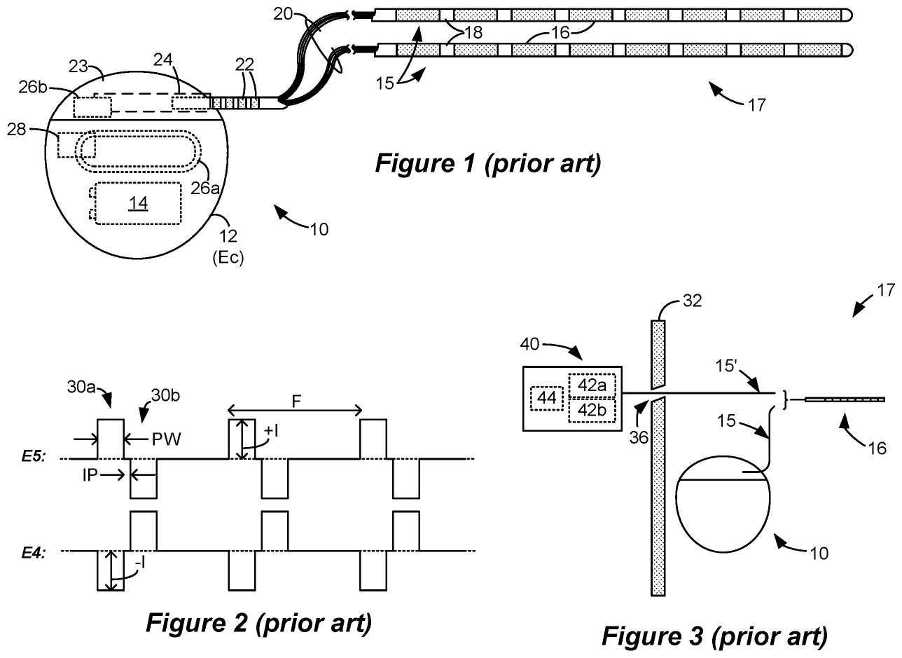

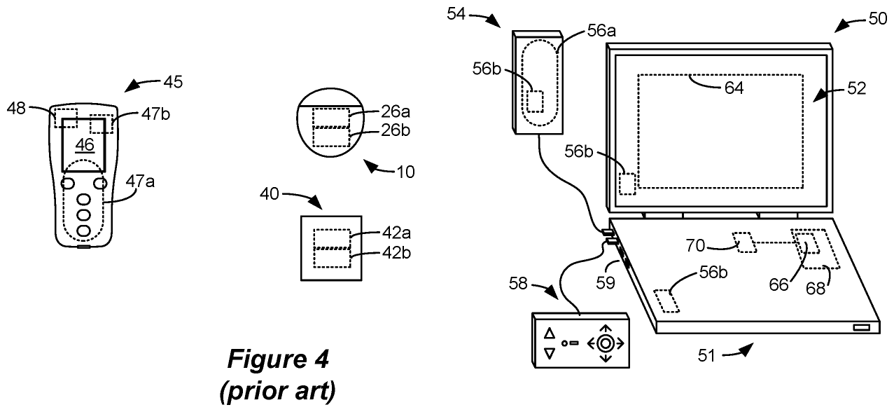

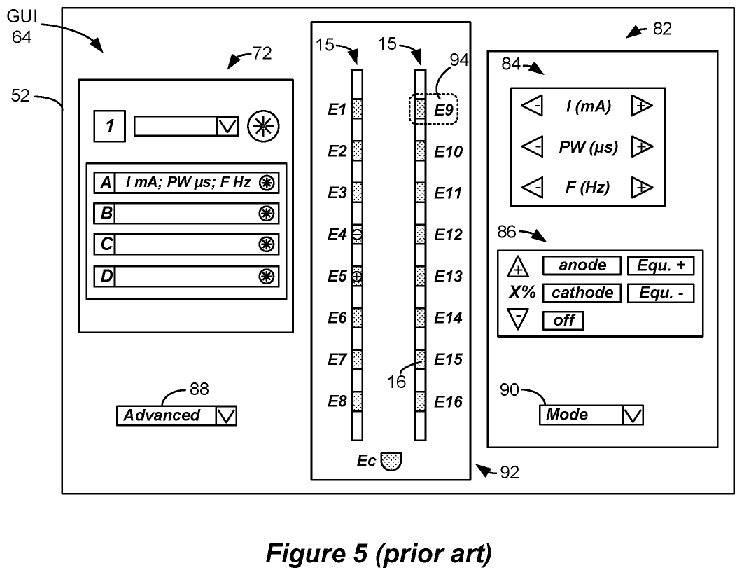

### DETAILED DESCRIPTION

A significant issue in stimulation therapy, and Spinal Cord Stimulation (SCS) therapy in particular, is determining the stimulation parameters that are best able to treat a patient's symptoms. As noted above, SCS stimulation parameters can include features like the amplitude of stimulation (I), the pulse width (PW) of the stimulation pulses, and the frequency (F) at which the stimulation pulses are issued. Other stimulation parameters used to place the stimulation at a location in the electrode array 17 to best treat the patient's symptoms are also important, and can include the electrodes that are active to form the stimulation (E), the polarity (P) of those active electrodes, and a percentage (X) indicating an relative amount of the amplitude each active electrode should receive. As explained above, these parameters define the location of poles in the electrode array 17.

The clinician programmer 50 is typically used to set these stimulation parameters, at least initially. Thereafter, a patient using his patient external controller 45 may also adjust at least some of the stimulation parameters, although perhaps not all of them. At a minimum, the external controller 45 usually permits the patient to adjust the amplitude (I) of the stimulation. This is sensible, because the stimulation may need to be changed depending on what the patient is doing. Patient activities (e.g., running, sleeping, etc.) and postures (e.g., standing, supine, prone, etc.) can affect the effectiveness of the stimulation therapy. For example, if a particular activity or posture moves the electrodes further from the spinal cord, it may be reasonable to increase the amplitude of the stimulation. Similarly, if a particular activity or posture moves the electrodes closer to the spinal cord, it may be reasonable to decrease the amplitude of the stimulation. Adjusting the amplitude can also be reasonable for other reasons. For example, the passage of time can cause changes in the electrical environment of the IPG (e.g., the formation of scar tissue or other factor that affect the coupling of the electrodes to the tissue). For these reasons, it is useful to allow the patient to adjust the amplitude to counteract these effects and to restore stimulation therapy to an effective level.

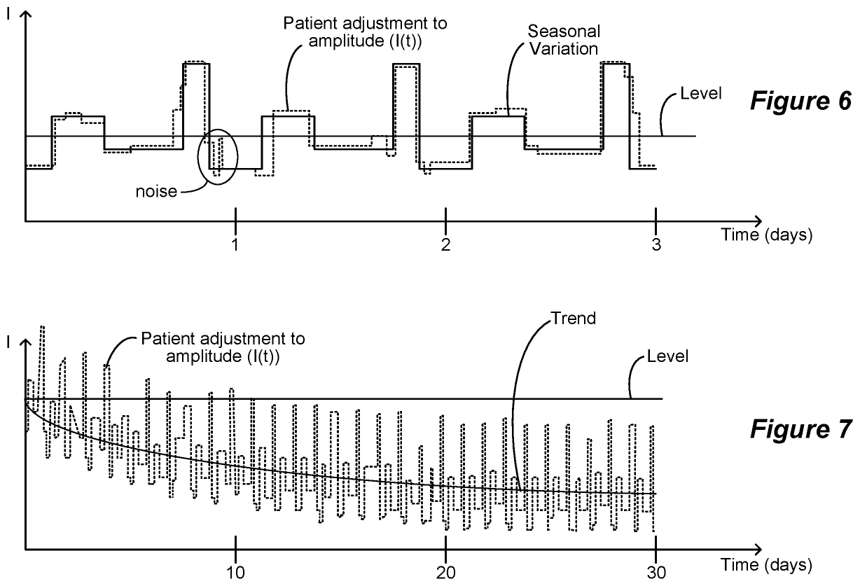

However, needing to constantly adjust the amplitude of stimulation can be burdensome on the patient. FIG. 6 shows in dotted lines an example of patient adjustment to amplitude I as a function of time (I(t)). Three days' worth of adjustments are shown. The amplitude I the patient has prescribed does not follow a perfect pattern. Nevertheless, it is also clear that amplitude I varies somewhat predictably, in particular over the course of a day. This makes sense, because a patient likely follows a somewhat predictable routine during the day, and this is reflected in the amplitudes the patient has selected. For example, at night when the patient is sleeping, the patient may set the amplitude to a relatively low amount. By contrast, higher amplitudes may be used during the day or evening when the patient may be walking or exercising. During office hours, when the patient may be sitting sit, the amplitude may be at a medium level. In short, it may be noticed from FIG. 6 that amplitude I(t) has on average a seasonal (e.g., periodic) variation over the course of a day, as shown in the solid lines. Obviously, the patient does not always use an amplitude consistent with this average seasonal variation; periods of “noise” may be noticed when these two differ, which are presumably the result of the patient engaging in a different activity or posture on that particular day. One skilled will realize that a number of different seasonal variations occurring over different time periods may be present in the patient-prescribed amplitude data, I(t). For example, although not shown, a time period of a week may reflect another seasonal variation, in which (for example) higher amplitudes are generally used over the week days when the patient is more active, and lower amplitudes used are generally used over the weekends when the patient is less active.

FIG. 7 shows patient adjustments to amplitude I(t) over a longer time period, such as a month (30 days). Again, the seasonal variation in the amplitude data over the course of each individual day is generally noticeable, although the amplitudes over each day may also vary somewhat, as explained with respect to FIG. 6. However, one can notice also a general trend in the amplitude data, in particular that the patient-prescribed amplitude is generally decreasing over time. This is something that has been noticed empirically by the inventors, and particularly when sub-perception stimulation is used. Sub-perception stimulation is explained further in Int'l (PCT) Application Publication No. WO 2021/178105, which is incorporated by reference in its entirety. Sub-perception stimulation is briefly explained. If the amplitude of the stimulation is above a perception threshold (pth, expressed for example in mA), the patient will feel the stimulation as paresthesia. If the amplitude of the stimulation is below pth, the patient will not feel the stimulation, although the stimulation still provides therapeutic benefit and symptom (e.g., pain) reduction. As explained in the '105 Publication, it is hypothesized that sub-perception stimulation may have a curative effect, meaning that less stimulation (less charge) is required over time, and this is reflected in the adjustments the patient has made to amplitude in FIG. 7, and in the smooth decrease reflected by the trend line. As noted above, electrode-to-tissue coupling may also change over time, which may manifest as a trend. In other examples, the trend line may also increase with time. For example, scar tissue may form over time which increases tissue resistance, which may require an increase in amplitude. Also, if the electrode array migrates (moves) in the patient (relative to the spinal cord), the location in the electrode array at which the stimulation is provided may gradually move away from tissue requiring therapeutic stimulation, which could also warrant increasing the amplitude over time to recruit that tissue with the stimulation.

As noted above, it may be burdensome to the patient to have to constantly adjust the amplitude of stimulation to address various changes (e.g., patient activity or posture, curative effects, formation of scar tissue, etc.), and as such the inventors see benefit in allowing the stimulation system to make automatic adjustments to the stimulation. While automatic adjustment to the amplitude would be most beneficial and is discussed herein as a primary example, automatic adjustment to other stimulation parameters (e.g., PW, F, or mean charge applied to the patient) may be beneficial as well.

In furtherance of this goal, the inventors propose a solution in which a time series analysis algorithm is used in one example to analyze adjustments a patient has made to the amplitude of stimulation I(t), and to use these previous adjustments to predict how the patient would likely adjust the amplitude in the future, i.e. to predict future amplitudes for the patient as a function of time, I′(t). Preferably, the algorithm determines one or more of an amplitude level, at least one seasonal variation, or at least one trend when predicting the amplitude I′(t). This predicted amplitude can then be used to automatically adjust the amplitude of the stimulation provided by the patient's IPG 10 or ETS 40. In certain examples, the algorithm may only use previous amplitude adjustments to predict the amplitude. In other examples, the algorithm may receive inputs beyond previous amplitude adjustments to assist in amplitude prediction. Such other inputs may include various parameters objectively or subjectively measured in the stimulator system, various models, patient information, and the like, as explained further below.

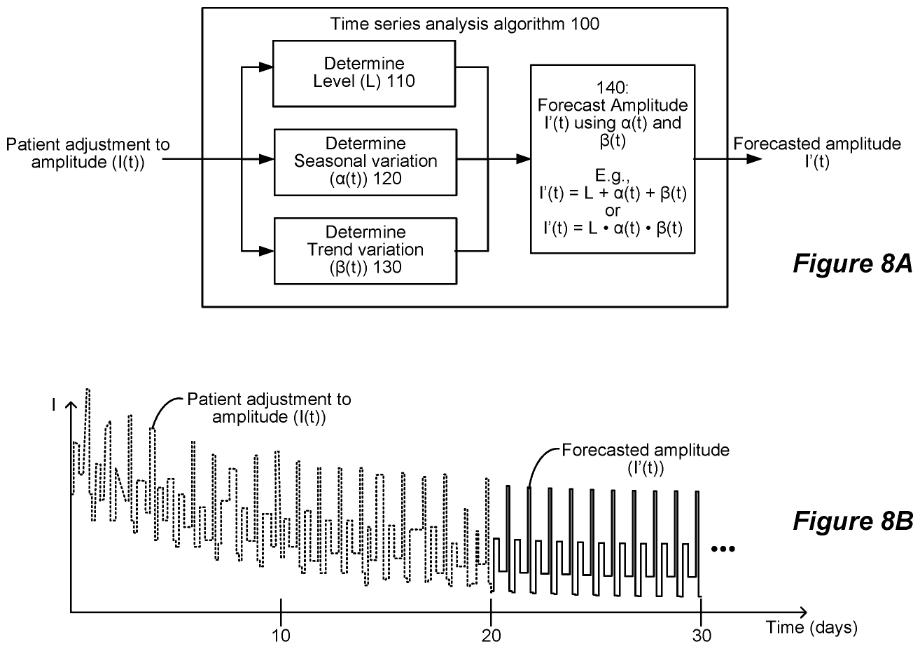

FIG. 8A shows a first example of a time series analysis algorithm 100. As shown, the algorithm 100 receives as an input previous adjustments the patient has made to the amplitude, I(t). This input is represented as an amplitude as a function of time, but one skilled will realize that this does not have to be a continuous function. Instead, I(t) may be represented as a series of points (t,I). For example, I(t) may comprise only points where amplitude I has been changed at a time t. As discussed with respect to FIGS. 6 and 7, I(t) will likely reflect a level of the stimulation, one or more seasonal variations, and a trend, and these aspects are determined in the algorithm 100 using modules 110-130. Specifically, module 110 determine the level L; module 120 determines the one or more seasonal variations α; and module 130 determines the trend β in the I(t) data. In this example, the level L is a constant (time-invariant) value generally indicative of an amplitude magnitude that is effective for the patient. The seasonal variation can be represented mathematically as a function of time α(t) over a relevant period in which the I(t) data shows periodicity, such as a day, week, year, etc. The trend data can also be represented as a function of time β(t), and unlike the seasonal variations would not be expected to have periodicity. Essentially, the trend explains how the amplitude magnitude for the patient generally deviates from the level over time.

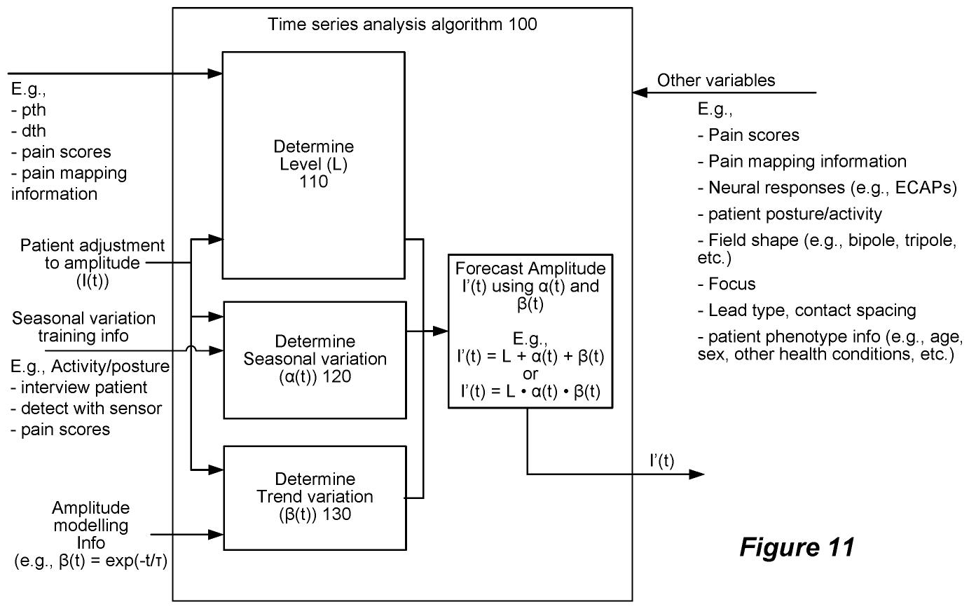

These aspects L, α(t), and β(t) as determined by modules 110-130 can be processed in a forecasting module 140, which determines a forecasted amplitude I′(t) for the patient based at least on the patient's previous amplitude adjustments I(t); further inputs to the algorithm 100 may also assist in forecasting I′(t) as explained further below with reference to FIG. 11. The forecasting module 140 can process these aspects in different ways, which may depend on the manner in which they were determined by modules 110-130. For example, and as shown in FIG. 8A, these aspects can be added (L+α(t)+β(t)) or multiplied (L*α(t)·β(t)), to determine the forecasted amplitude I′(t).

There are numerous ways in which time series analysis algorithm 100 can be implemented. For example, frequency domain techniques such as Fourier transforms may be used. In a preferred example, a seasonal autoregressive integrated moving average (SARIMA) algorithm can be used, which are well known. In this regard, note that a particular time series algorithm may not discretely calculate aspects L, α(t), and β(t) as illustrated above. Nevertheless, it is still useful to envision the forecasted amplitude I′(t) in this way for illustration purposes.

FIG. 8B shows an example of the use of the time series analysis algorithm 100. In this example, patient adjustments to amplitude I(t) are reviewed over a 20-day period, although this period is variable depending on the algorithm 100 used. As discussed above, notice that these amplitude adjustments have some randomness (noise) but also illustrate noticeable patterns. The algorithm 100 has processed I(t) to forecast the amplitude I′ (t) the patient will require over a future time period, such as 10 days in this example, although again this period is variable. Notice that processing tends to remove noise from the forecasted amplitude I′(t), and establishes (average) seasonal and trend variations.

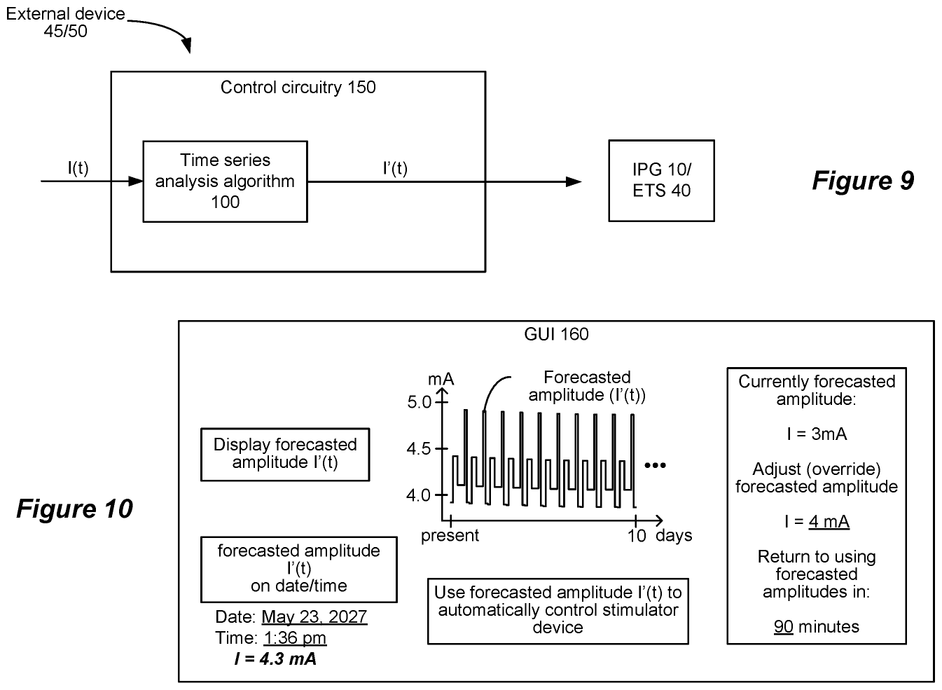

FIG. 9 shows how the forecasted amplitude I′(t) can be used to automatically control stimulation in a patient stimulator device, such as an IPG 10 or ETS 40. In this example, the time series analysis algorithm 100 is programmed into control circuitry 150 of an external device that has access to previous amplitude adjustments I(t). In one example, the control circuitry 150 can comprises a microcontroller or microprocessor such as those made by Intel Corporation. See, e.g., ww.intel.com. In a preferred example, the external device would comprise the patient external controller 45 used by the patient to adjust the amplitude. However, the external device could also comprise any external device capable of receiving and processing data I(t), such as the clinician programmer 50. I′(t) can be transmitted to the IPG 10 or ETS 40 to automatically adjust the amplitude of the stimulation provide to the patient, using telemetry circuitry in the external device. Again, this is most preferably done using the patient external controller 45, which as noted earlier can be used by the patient to adjust the amplitude I. Automatic control can occur in different ways. For example, the external device can automatically send an instruction to the IPG 10 or ETS 40 to change the amplitude only at those times when I′(t) is forecasted to change. Alternatively, I′(t) may be sent to the IPG 10 or ETS 40 at one time as a complete data set, leaving it to these devices to determine when the amplitude should be adjusted per I′(t).

The time series analysis algorithm 100 in the system is preferably accompanied by the use of a graphical user interface (GUI) 160 displayable the external device in which algorithm 100 operates, as shown in FIG. 10. For example, the GUI 160 can include a selectable option to allow the patient to automatically control his stimulator device using the forecasted amplitude I′(t). This can involve transmitting I′(t) to the stimulator device, which can occur in different ways as discussed above with respect to FIG. 9. Note that control of the stimulator device via the algorithm 100 can occur automatically, and GUI 160 doesn't necessarily have to have a selectable option to affect this.

The GUI 160 also allows the patient to review information relevant to operation of the algorithm 100. For example, a user can review the forecasted amplitude I′(t), which may be displayed graphically as shown. The patient may also evaluate what the forecasted amplitude will be at a particular point in time, and thus GUI 160 may include options to enter a particular day and time, and output the forecasted current at that point in time. GUI 160 may also be used to provide other details about use or operation of the algorithm 100. For example, although not shown, the GUI 160 could display options to review information relevant to a statistical confidence of the forecasted amplitude I′(t), to glean a sense of how reliable the forecasted amplitude I′(t) is expected to be.

Automatic amplitude control via I′(t) doesn't necessarily exclude the use of occasional manual adjustment of the amplitude by the patient, and GUI 160 can provide for such manual control and otherwise allow the patient to override (at least temporarily) use of the algorithm 100 for automatic control. While I′(t) attempts to forecast an amplitude that a patient may need at a particular point in time, this amplitude may not be optimal, especially if the patient unpredictably engages in an activity. For example, the algorithm 100 may predict that a patient should currently receive an amplitude of 3 mA, as reflected in the currently-forecasted amplitude shown in FIG. 10. This may be a relatively low value for the patient, for example because the patient is normally sitting still at this time. If the patient (unpredictably) decides to go running at this time, the patient may wish to manually increase the amplitude, say to 4 mA as shown in FIG. 10. In this sense, the patient can override use of the algorithm 100 and use of the amplitude that I′(t) predicts at this time. After a period of manual amplitude adjustment, the patient may use the GUI 160 to revert back to use of the algorithm 100 to once again provide automatic amplitude adjustment. In the example shown, the patient can set a period of time (e.g., 90 minutes) after which such reversion should take place, although automatic reversion after a time period could also occur. Note that manual adjustments to the amplitude can continue to be assessed by the time series analysis algorithm, which in turn can update the forecasted amplitude I′ (t) as necessary.

Use of time series analysis algorithm 100, and using the determined forecasted amplitude I′(t) for automated control of the amplitude is expected to be convenient for the patient. Even though the patient can and should manually control the amplitude from time to time, and as such may override automated control as described above, the automatic adjustments will also from time to time preclude the need for the patient to make adjustments, thus conveniencing the patient. Automatic adjustment may also provide other system benefits, such as power savings. Further, time series analysis algorithm 100 doesn't require in the system the sensing of information beyond previous amplitude adjustment (such as detecting a patient posture or activity) to perform its function of forecasting amplitude adjustment I′(t). That being said, sensing certain information and providing that sensed information to the algorithm 100 as inputs can be useful, as described further below.

FIG. 11 shows modifications that can be made to the time series algorithm 100. To this point it is has assumed that the forecasted amplitude I′(t) is determined solely on the basis of previous adjustments to the amplitude I(t) that the patient has made. However, the algorithm 100 may consider other inputs, with such inputs being used exclusively or in addition to I(t) to determine I′ (t). Certain of these other inputs may only affect one of the modules 110, 120, or 130, and hence may only affect level L, seasonal variation α(t), and trend variation β(t).

FIG. 11 shows some examples of inputs that can be used to adjust the level L, which are input to level module 110. These inputs can comprise measurement that may be taken from time to time. For example, the perception threshold pth—e.g., the lowest amplitude at which the patient can feel stimulation, or the highest amplitude where the patient cannot feel the stimulation—can be measured. Such measurements can be made by the physician (e.g., using clinician programmer 50), or by the patient using his patient external controller 45. (Note that the controller 45 may prompt the patient to make periodic pth measurements). Similarly, a discomfort threshold dth—e.g., the highest amplitude the patient can tolerate—can also be measured. A shift in these values would tend to suggest that the amplitude should change as well.

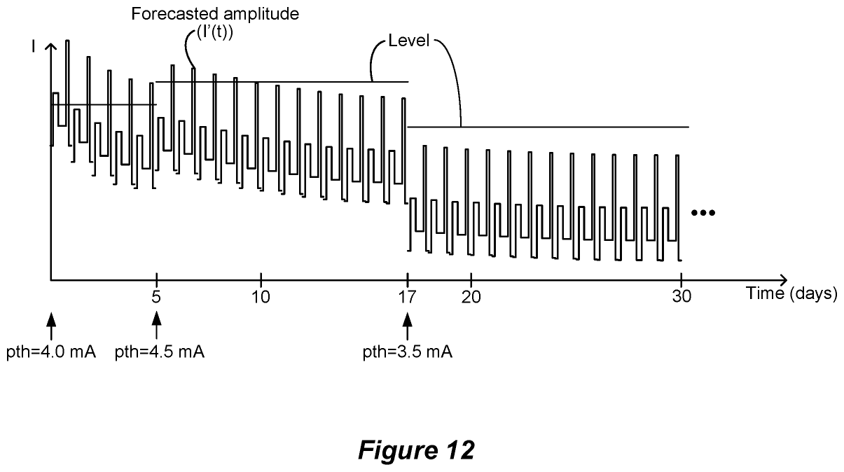

For example, consider the example of FIG. 12, which shows how pth measurements can be used to modify the level L (per module 110) and hence the forecasted amplitude I′(t). Here, the algorithm 100 has previously operated to predict a forecasted amplitude I′(t), in accordance with a level, and seasonal and trends variations. At the time of the prediction, the patient's paresthesia threshold pth was measured to be 4.0 mA. Assume at day 5 the measured pth changes, to 4.5 mA. This change in pth would suggest that the patient requires higher amplitudes, and hence that the forecasted amplitude I′ (t) should be increased by the algorithm 100 (or module 110 more specifically). This raises the forecasted amplitudes I′(t) upwards at day 5. However, seasonal (periodic) and trend variations may otherwise be unaltered, and continue as predicted earlier. Assume at day 17 the measured pth changes to 3.5 mA. This change in pth would suggest that the patient requires lower amplitudes, and hence that the forecasted amplitude I′(t) should be decreased at day 5. Dth would also vary with required amplitudes, and could also have been used in this example to raise or lower the level L of I′(t). FIG. 12 is just example of an input that can be used to adjust the level determined by the algorithm 100. In another example, NRS pain scores (from 1 to 10), or pain mapping information may also be used to affect the level L. An example of pain mapping information can comprise a percentage estimation by the patient as to how well stimulation seems to be addressing or “covering” his pain. If pain scores increase (suggesting more pain), or if pain mapping information worsens (e.g., percentage of coverage dropping), the algorithm 100 may raise level L to increase the forecasted amplitude I′(t).

Other inputs may be used to modify determination of the seasonal variation α(t) (in module 120) and the trend β(t) (in module 130). For example, seasonal variation information may be provided to module 120, which may comprise information initially used to train module 120 in accordance with patient activity and/or posture patterns. For example, such training information can be gleaned from a patient interview, where a patient is asked what kind of activity he is typically engaged in at particular times of days. This time schedule can be provided to the seasonal variation module along with information that converts the activities to expected amplitude values. For example, more strenuous activities or postures (exercise, walking, standing, etc.) can be converted to higher predicted amplitudes (e.g., 10% above baseline), and lower strenuous activities and postures (sitting, sleeping, etc.) can be converted to lower predicted amplitudes (e.g., 10% below baseline), thus allowing an initial α(t) to be established. In another example, a patient can wear a sensor that detects activity and posture as a function of time, such as a sensor that includes an accelerometer. The sensor may be worn for a training period (e.g., one week), and detected activity or postures averaged over the course of the day. Again, detected activity can be converted to predicted amplitudes (more strenuous, higher amplitude; lower strenuous, lower amplitude) and provided to seasonal module 120. In another example, therapy ratings such as pain scores can be tracked as a function of time, such as by having the patient enter such scores into his external controller 45. Predicted amplitudes may also be determinable using these pain scores entered into the system over a period of time. For example, if a patient's pain score is high at a given time, it may be assumed that his currently-used amplitude is too low; if the pain score is low, perhaps the currently-user amplitude unnecessarily high. From any of these examples, a predicted amplitude as a function of time over a period (e.g., 1 day) can be developed (α(t)), and provided to the seasonal module 120. While such seasonal variation training information could be solely used by the algorithm 100, it is preferred that such training information merely acts as a starting point for the algorithm 100. As time goes on and the patient continues to make amplitude adjustment I(t), the algorithm 100 (particularly module 120) can adjust or update seasonal variation α(t) as necessary.

In another example, amplitude modeling information may be provided to the trend module 130. Such modeling information may be empirically determined from a number of patients, and may reflect how a trend in amplitudes typically changes for patients as a function of time. In one example, such modeling information may suggest that the trend β(t) should decrease exponentially in accordance with a time constant of τ (i.e., β(t)=exp(−t/τ)). The trend module may therefore tend to decrease the forecasted amplitude accordingly, at least initially. Of course, this trend may also be modified (i.e., τ) depending on trends noticed in the patient amplitude adjustments I(t).

FIG. 11 shows still other inputs that could be used by the time series analysis algorithm 100, which may be used by one or more of modules 110-130, and/or by forecasting module 140. Such inputs may comprise other measured values, which may be objectively or subjectively measured. An example of a subject measurement may comprise pain score, such as a NRS pain score (a 1-10 ranking). Pain mapping information—such as how completely stimulation seems to be covering a patient pain, and/or the location of that pain—can also be subjectively measured, as explained further in the above-incorporated '105 Publication. Objective measurements are measured by the IPG or ETS 140, or by external equipment. For example, neural responses to stimulation can be objectively measured, such as Evoked Compound Action Potentials (ECAPs), as explained for example in U.S. Pat. No. 10,406,368. Patient activity or posture can also be measured, using for example an accelerometer in the IPG 10.

Still other inputs to the time series analysis algorithm 100 can include parameters that affect the stimulation field generated in the patient by the stimulation. For example, the field shape can be considered, such as whether the stimulation is produced in the electrode array as a bipole (with one anode and one cathode); as a tripole (with for example two anode poles flanking a central cathode pole), or other pole configurations. The “focus” or distance between those poles can also comprise an input to algorithm 100, as can the physical spacing between the electrodes and the type of leads used in the electrode array.

Lastly, patient phenotype information may be considered by the time series analysis algorithm 100 as well, including a patient's age, sex, information concerning patient vitals, disease diagnosis, other health-related parameters.

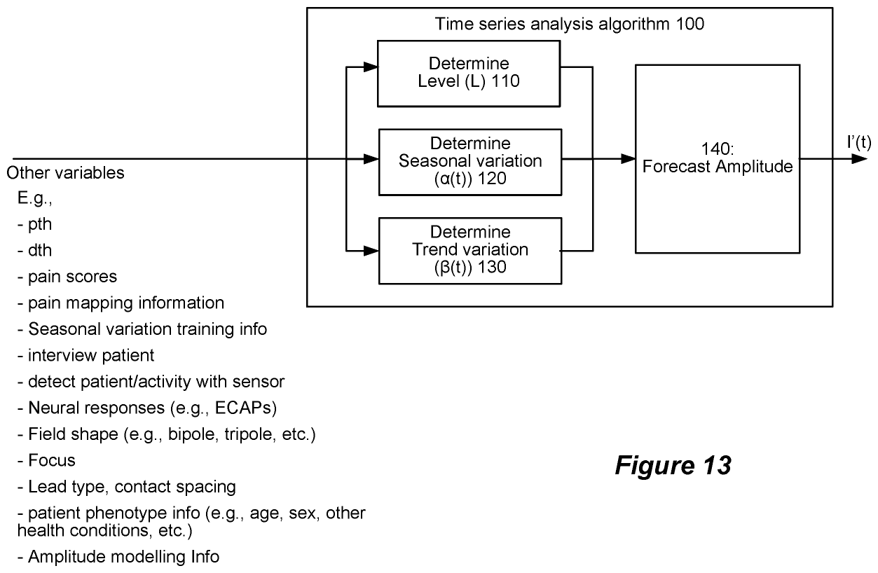

In FIG. 11, a forecasted amplitude I′(t) is determined using both patient adjustments to amplitude I(t) as well as other variables. However, this isn't strictly required. FIG. 13 shows yet another example of time series analysis algorithm 100, in which the forecasted amplitude I′(t) can be determined by the algorithm 100 even without consideration of previous amplitude adjustments I(t). Instead, only other variables such as those mentioned earlier (pth, pth, pain scores, etc.) are used to predict I′(t). In this example, it is assumed that these other variables are time variable, and are entered into the algorithm 100 at a suitable frequency to be able to determine I′(t) with sufficient resolution, including both seasonal (α(t)) and trend (β(t)) components.

One skilled will understand that the time series analysis algorithm 100 described herein can be formulated and stored as instructions in a computer-readable media, such as in a magnetic, optical, or solid state memory. The computer-readable media with such stored instructions may reside with a relevant external device, such as the external controller 45 or clinician programmer 50, in a memory stick used to transmit information to such devices, or in the IPG 10 or ETS 40. The computer-readable media may also reside in a server or any other computer device, thus allowing instructions to be downloaded to these stimulator system devices, via the Internet for example.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method using an external device for controlling a stimulator device that provides stimulation to a patient, the method comprising:
receiving at the external device adjustments to a stimulation parameter of the stimulation provided by the stimulator device;
analyzing at the external device the adjustments to the stimulation parameter to determine a forecasted stimulation parameter, wherein the forecasted stimulation parameter comprises a periodic variation as a function of time or a non-periodic trend as a function of time; and
automatically controlling the stimulator device over time to provide the stimulation in accordance with the forecasted stimulation parameter.

2. The method of claim 1, wherein analyzing the adjustment to the stimulation parameter comprises use of a time series analysis algorithm.

3. The method of claim 2, wherein the time series analysis algorithm comprises a seasonal autoregressive integrated moving average algorithm.

4. The method of claim 1, wherein the adjusted stimulation parameter and the forecasted stimulation parameter comprise a stimulation amplitude.

5. The method of claim 1, wherein the forecasted stimulation parameter comprises a sum of the periodic variation and the non-periodic trend.

6. The method of claim 1, wherein the forecasted stimulation parameter comprises a product of the periodic variation and the non-periodic trend.

7. The method of claim 1, wherein the forecasted stimulation parameter further comprises a time-invariant level.

8. The method of claim 7, wherein the forecasted stimulation parameter comprises a function of the periodic variation, the non-periodic trend, and the time-invariant level.

9. The method of claim 1, wherein the external device comprises a portable patient external controller.

10. The method of claim 1, wherein the forecasted stimulation parameter comprises the periodic variation as a function of time and the non-periodic trend as a function of time.

11. The method of claim 1, wherein the external device transmits the forecasted stimulation parameter to the stimulator device to automatically control the stimulator device in accordance with the forecasted stimulation parameter.

12. The method of claim 1, wherein the stimulator device is automatically controlled in accordance with the forecasted stimulation parameter by adjusting that stimulation parameter at the stimulator device as a function of time.

13. The method of claim 1, wherein the adjustments to the stimulation parameter are received at a user interface of the external device.

14. The method of claim 13, wherein the user interface comprises a selectable option to cause the external device to automatically control the stimulator device to provide the stimulation in accordance with the forecasted stimulation parameter.

15. The method of claim 13, further comprising overriding automatic control of the stimulator device by receiving at least one input at the user interface to manually control the stimulation parameter.

16. The method of claim 15, further comprising reverting to automatic control of the stimulator device a time period after manual control of the stimulation parameter.

17. The method of claim 1, wherein the stimulator device provides the stimulation as electrical stimulation at one or more electrodes of the stimulator device.

18. The method of claim 1, wherein the stimulator device is implantable in the patient.

19. A system, comprising:
an external device for controlling a stimulator device that provides stimulation to a patient, the external device comprising:
a user interface configured to receive adjustments to a stimulation parameter of the stimulation provided by the stimulator device; and
controller circuitry programmed with an algorithm, wherein the algorithm is configured to:
analyze the adjustments to the stimulation parameter to determine a forecasted stimulation parameter, wherein the forecasted stimulation parameter comprises a periodic variation as a function of time or a non-periodic trend as a function of time, and
automatically control the stimulator device over time to provide the stimulation in accordance with the forecasted stimulation parameter.

20. A non-transitory computer readable medium comprising instructions executable on an external device for controlling a stimulator device that provides stimulation to a patient, wherein the instructions when executed enable the external device to:
receive adjustments to a stimulation parameter of the stimulation provided by the stimulator device;
analyze the adjustments to the stimulation parameter to determine a forecasted stimulation parameter as a function of time, wherein the forecasted stimulation parameter comprises a periodic variation as a function of time or a non-periodic trend as a function of time; and
automatically control the stimulator device over time to provide the stimulation in accordance with the forecasted stimulation parameter.
